(19) United States
(12) Patent Application Publication
(10) Pub. No.: US 2009/0054368 A1
(43) Pub. Date: Feb. 26, 2009

(54) Substituted gemini surfactant compounds

(75) Inventors: Shawn Wettig, Saskatoon (CA); Ronald Ernest Verrall, Saskatoon (CA); Marianna Foldvari, Kitchener (CA)

Correspondence Address: King & Spalding LLP, P.O. Box 889, Belmont, CA, 94002-0889, US

(73) Assignee: University of Saskatchewan, Saskatoon (CA)

(21) Appl. No.: 12/215,963

(22) Filed: Jul. 1, 2008

Related U.S. Application Data
(60) Provisional application No. 60/958,563, filed on Jul. 6, 2007.

(51) Int. Cl. A61K 31/711; A61K 47/06; A61K 47/16; C12Q 1/68

(52) U.S. Cl. 514/44; 514/772; 514/788; 435/6

(57) ABSTRACT

The compositions, systems, and methods relate to substituted and asymmetric gemini surfactants for use in delivering biologically active agents, such as nucleic acids, to a subject. The described compositions, systems, and methods are useful for gene therapy.

### PRIORITY

The present application claims priority to U.S. Provisional Application Ser. No. 60/958,563, filed on Jul. 6, 2007, which is herein incorporated by reference in its entirety.

### GOVERNMENT FUNDING

Funding was provided by the Natural Sciences and Engineering Research Council of Canada (NSERC), the Canadian Institutes of Health research (CIHR), and a fellowship from the Saskatchewan Synchrotron Institute. Use of the National Synchrotron Light Source, Brookhaven National Laboratory, was supported by the U.S. Department of Energy under Contract No. DE-AC02-98CH10886. Accordingly, the Canadian and/or United States government(s) may retain certain rights in the present invention.

### TECHNICAL FIELD

The compositions, systems, and methods relate to spacer-substituted and asymmetric gemini surfactants for use in delivering biologically active agents, such as nucleic acids, to a subject. The described compositions, systems, and methods are useful for gene therapy.

### BACKGROUND

Gene therapy is the delivery of nucleic acids encoding proteins of interest to preselected cells in a subject, wherein the subject synthesizes the protein of interest using exogenous translation machinery. Several methods for delivering nucleic acids to the cells of a subject have been described, including the use of viral vectors, ballistic delivery of naked DNA, lipid-mediated transfection, and the like.

Viral vectors are currently the most efficient nucleic acid delivery vehicles, representing more than 65% of ongoing clinical trials; however, they also suffer from several disadvantages (reviewed in [3, 4]). For example, the nature of the viral capsid has the potential to generate a severe immune response, as has been demonstrated in a number of animal and human trials [5]. Additionally, the loading capacity of viral vectors is limited to DNA of 2-3 kb in size, there are concerns regarding long-term storage, and preparation and purification must follow rigorous protocols.

Non-viral liposome-based nucleic acid delivery vehicles have low toxicity, are non-immunogenic, do not suffer from limitations on the size of plasmid DNA that can be encapsulated, and allow for specialized delivery options (such as targeted delivery, time-dependent release, enhanced circulation times, etc.) [6], all of which have been documented. The main disadvantage in using non-viral delivery vehicles is low in vivo transfection efficiency.

The compositions and methods to be described represent a novel approach for delivering biologically active agents, such as nucleic acids, using rationally-designed surfactants.

### REFERENCES

Each of the following references, and additional references cited herein, is incorporated by reference in its entirety.
1. Pearson, S. et al. Nat Biotechnol 2004; 22:3-4.
2. Branca, M. et al. Nat Biotechnol 2005; 23:519-21.
3. Gardlik, R. et al. Med Sci Monit 2005; 11:RA110-121.
4. Patil, S. Aaps J. 2005; 7:E61-77.
5. Goncalves, M. et al. Bioessays. 2005; 27:506-17.
6. Smyth T. et al. Curr Med Chem 2003; 10:1279-87.
7. Hulst, R. et al. Eur J Org Chem 2004: 835-849.
8. Niculescu-Duvaz, D. et al. Curr Med Chem 2003; 10:1233-61.
9. McGregor, C. et al. J Am Chem Soc 2001; 123:6215-20.
10. Bombelli, C. et al. J Med Chem 2005; 48:5378-82.
11. Badea, I. et al. J Gene Med 2005; 7:1200-14.
12. Rosenzweig, H. Bioconjug Chem 2001; 12:258-63.
13. Kirby, A. et al. Angew Chem Int Edit 2003; 42:1448-57.
14. Bell, P. et al. J Am Chem Soc 2003; 125:1551-58.
15. Bergsma, M. et al. J Colloid Interf Sci 2001; 243:491-95.
16. Fielden, M. et al. Eur J Biochem 2001; 268:1269-79.
17. Johnsson, M. et al. Langmuir 2003; 19:4609-18.
18. Johnsson, M. et al. J Am Chem Soc 2003; 125:757-60.
19. Johnsson, M. et al. J Phys Org Chem 2004; 17:934-44.
20. Yoshimura, et al. Langmuir 2005; 21:10409-15.
21. Vijayanathan, V. et al. Nucleic Acids Res 2004; 32:127-34.
22. Vijayanathan, V. et al. Biochemistry 2001; 40:13644-51.
23. Wilson, R. et al. Biochemistry 1979; 18:2192-96.
24. Wettig, S. et al. Phys Chem Chem Phys 2007; (Advance Article): DOI: 10.1039/b613269c.
25. Wettig S, and Verrall R. J Colloid Interf Sci 2001; 235:310-16.
26. Balendiran, K. et al. Acta Crystallogr D Biol Crystallogr 1995; 51:190-98.
27. Dass, C. Int J Pharm 2002; 241:1-25.
28. Koltover, I. et al. Science 1998; 281:78-81.
29. Radler, J. et al. Science 1997; 275:810-14.
30. Ewert, K. et al. Curr Med Chem 2004; 11:133-49.
31. Chesnoy, S. et al. Annu Rev Biophys Biomol Struct 2000; 29:27-47.
32. Hirsch-Lerner, D. et al. Biochimica et Biophysica Acta (BBA)—Biomembranes 2005; 1714:71-84.
33. Simberg, D. et al. J Biol Chem 2001; 276:47453-59.
34. Zuidam, N. et al. FEBS Lett 1999; 457:419-22.
35. Braun, C. et al. Biophys J 2003; 84:1114-23.
36. Saenger, W. Principles of Nucleic Acid Structure. New York: Springer-Verlag, 1984.
37. Ruiz-Chica J. et al. Biophys J 2001; 80:443-54.
38. Ouameur, A. and Tajmir-Riahi, H. J Biol Chem 2004; 279:42041-54.
39. Tycko, B. and Maxfield, F. Cell 1982; 28:643-51.
40. Pitard, B. Somat Cell Mol Genet. 2002; 27:5-15.
41. Lin, A. et al. J Drug Target 2000; 8:13-27.
42. Kreiss, P. et al. Nucleic Acids Res 1999; 27:3792-98.
43. Zhou, S. et al. Biomacromolecules 2004; 5:1256-61.
44. Lin, A. et al. Biophys J 2003; 84:3307-16.
45. Rosenzweig, H. et al. Bioconjug Chem 2000; 11:306-13.
46. Foldvari, M. et al. NSTI Nanotech 2006, NSTI Nanotechnology Conference and Trade Show, Boston, Mass., United States, May 7-11, 2006 2006; 2:400-03.
47. Foldvari, M. et al. J Exp Nanosci 2006; 1:165-76.
48. Angelov, B. et al. Langmuir 1999; 15: 8225-34.
49. Boal, A. and Rotello, V. J. Am. Chem. Soc., 2002, 124:P5019.
50. Nakamura, F. and Hara, M. Mol. Cryst. Liq. Cryst., 2002; 377:57.
51, Wettig, S. et al. Langmuir, 2002; 18:5354.
52. Grosmaire, L. et al. J, Colloid. Int. Sci., 2002; 246:75.
53. Huang, H. and Verrall, R. Can J. Chem., 1997; 75:1445.

### SUMMARY

In some aspects, a delivery system for a biologically active agent is provided comprising:

a gemini surfactant in admixture with a biologically active agent,

said gemini surfactant having head groups and a spacer linking said head groups, said spacer comprising a hydrophilic substituent in the spacer, wherein the delivery system, when in contact with skin or a mucosal membrane, provides a therapeutic effect.

In some embodiments, the hydrophilic substituent is selected from the group consisting of aza, imino, hydroxyl, and ether. In some embodiments, the hydrophilic substituent is the result of an N-substitution or an O-substitution. In some embodiments, the spacer is N-substituted and the substituent is selected from aza and imino.

In some embodiments, the spacer is from 3-8 atoms in length and comprises an imino substituent. In certain embodiments, the spacer is 7 atoms in length. In certain embodiments the gemini surfactant is 12-7NH-12. In certain embodiments the gemini surfactant is 1,9-bis(dodecyl)-1,1,9,9-tetramethyl-5-imino-1,9,-nonanediammonium dibromide.

In some embodiments, the spacer is from 3-8 atoms in length and comprises an aza substituent. In particular embodiments, the spacer is 7 atoms in length. In certain embodiments the gemini surfactant is 12-7N-12. In certain embodiments, the gemini surfactant is 1,9-bis(dodecyl)-1,1,5,9,9-pentamethyl-5-aza-1,9,-nonanediammonium dibromide.

In other embodiments, the spacer is O-substituted and the substituent is selected from hydroxyl and ether. In particular embodiments, the substituent is hydroxyl. In certain embodiments, the gemini surfactant is 12-4(OH)₂-12. In related embodiments, the substituent is ether. In certain embodiments, the gemini surfactant is 12-EO₁-12.

In some embodiments, the above delivery system is for use in the absence of added helper lipid. In other embodiments, the delivery system further includes DOPE.

In some embodiments, the gemini surfactant is a cationic surfactant. In particular embodiments, the gemini cationic surfactant is of a quaternary ammonium type. The gemini cationic surfactant may have a hydrophobic tail comprising a C3-C30 alkyl group, linear or branched, saturated or unsaturated.

In some embodiments, the biologically active agent is a nucleic acid. In particular embodiments, the agent is DNA.

In some embodiments, the delivery system is provided as a component of a pharmaceutical product. In related embodiments, the delivery system is formulated with a pharmaceutically acceptable component to form a pharmaceutical composition.

In some aspects, the above delivery system further comprises an asymmetric gemini surfactant having a first tail and first head group, a second tail and second head group, and a spacer linking said first and second head groups, wherein the first tail comprises a pyrene moiety. In particular embodiments, the asymmetric gemini surfactant is py-3-12. In particular embodiments, the asymmetric gemini surfactant is py-6-12.

In some embodiments, the asymmetric gemini surfactant is present in a trace amount to allow the detection of the gemini surfactant in admixture with the biologically active agent. In particular embodiments, the detection is performed by measuring fluorescence at 480 nm.

In another aspect, a delivery system for a biologically active agent is provided comprising:

an asymmetric gemini surfactant in admixture with a biologically active agent,

said asymmetric gemini surfactant having a first tail and first head group, a second tail and second head group, and a spacer linking said first and second head groups, wherein said first tail comprises a pyrene moiety,

wherein the delivery system, when in contact with skin or a mucosal membrane, provides a therapeutic effect. In particular embodiments, the asymmetric gemini surfactant is py-3-12. In other embodiments, the asymmetric gemini surfactant is py-6-12.

In some embodiments, the biologically active agent is a nucleic acid. In particular embodiments, the biologically active agent is DNA.

In some embodiments, the delivery system is provided as a component of a pharmaceutical product. In related embodiments, the delivery system is formulated with a pharmaceutically acceptable component to form a pharmaceutical composition.

In another aspect, a gemini surfactant is provided having head groups and a spacer linking said head groups, said spacer comprising an N-substituted substituent selected from aza and imino.

In some embodiments, the spacer is from 3-8 atoms in length and comprises an imino substituent. In particular embodiments, the spacer is 7 atoms in length. In certain embodiments, the gemini surfactant is 12-7NH-12. In certain embodiments, the gemini surfactant is 1,9-bis(dodecyl)-1,1,9,9-tetramethyl-5-imino-1,9,-nonanediammonium dibromide.

In some embodiments, the spacer is from 3-8 atoms in length and comprises an aza substituent. In some embodiments, the spacer is 7 atoms in length. In certain embodiments, the gemini surfactant is 12-7N-12. In certain embodiments, the gemini surfactant is 1,9-bis(dodecyl)-1,1,5,9,9-pentamethyl-5-aza-1,9,-nonanediammonium dibromide.

In another aspect, a gemini surfactant is provided having head groups and a spacer linking said head groups, said spacer comprising an O-substituted substituent selected from hydroxyl and ether. In some embodiments, the gemini surfactant is 12-4(OH)₂-12. In some embodiments, the gemini surfactant is 12-EO₁-12.

In another aspect, an asymmetric gemini surfactant is provided having a first tail and first head group, a second tail and second head group, and a spacer linking said first and second head groups, wherein the first tail comprises a pyrene moiety.

In some embodiments, the spacer is from 3-6 atoms in length. In particular embodiments, the spacer is 3 atoms in length. In certain embodiments, the gemini surfactant is py-3-12. In another particular embodiment, the spacer is 6 atoms in length. In particular embodiments, the gemini surfactant is py-6-12.

In another aspect, a method of treating skin disorders and metabolic diseases is provided, comprising:

contacting the skin or mucosal membrane of a subject with a delivery system comprising a gemini surfactant having head groups and a spacer linking said head groups, said spacer being N-substituted or O-substituted, in admixture with a biologically active agent in a topical formulation, wherein the delivery system, when in contact with skin or a mucosal membrane, provides a therapeutic effect.

In some embodiments, the gemini surfactant is a gemini cationic surfactant. In particular embodiments, the gemini cationic surfactant is of a quaternary ammonium type. In some embodiments, the gemini surfactant is selected from the croup consisting of 12-7NH-12, 12-7N-12, 12-4(OH)₂-12, and 12-EO₁-12.

In another aspect, a method for detecting a gemini surfactant-DNA complex in a subject, comprising:

adding to an admixture of a gemini surfactant and DNA an amount of an asymmetric, pyrene-substituted gemini surfactant,

exciting the pyrene-substituted gemini surfactant present in the admixture at a wavelength suitable for exiting the pyrene moiety, and

measuring the fluoresce of a the pyrene moiety,

whereby the presence of the asymmetric, pyrene-substituted gemini surfactant in the admixture allows the detection of the admixture in the subject. In some embodiments, the fluoresce of the pyrene moiety is measured at a wavelength of about 480 nm. In particular embodiments, the asymmetric, pyrene-substituted gemini surfactant is py-3-12. In other particular embodiments, the asymmetric, pyrene-substituted gemini surfactant is py-6-12.

### BRIEF DESCRIPTION OF THE DRAWINGS

FIGS. 1A-1G show energy minimized structures of exemplary spacer-substituted (A-E), asymmetric (F), and unsubstituted (G) gemini surfactants. FIG. 1A shows the structures of the 12-5N-12, 12-8N-12, 12-7N-12, and 12-7NH-12 surfactants. FIGS. 1B and 1C show the structures of two surfactants related to the 12-7NH-12 surfactant. FIGS. 1D and 1E show the structures of 12-3-12, 12-4(OH)₂-12 and 12-EO₁-12, respectively. FIG. 1F shows the general structure of an asymmetric surfactant. FIG. 1G shows the structure of the unsubstituted gemini surfactant 12-3-12.

FIG. 2A is a graph showing the results of transfection of COS-7 cells with plasmid DNA-N-substituted-spacer gemini surfactant complexes (PG, black bars) or plasmid DNA-gemini surfactant-DOPE nanoparticles (PGL, white bars); ρ_{+/-}=10 (n=6, error bars indicate standard deviation). Data is also presented for DC-Chol with (white) and without (black) DOPE, plasmid DNA with (white) and without (black) DOPE, and Lipofectamine (shaded).

FIG. 2B is a graph showing the results of transfection of cells with plasmid DNA-O-substituted-spacer gemini surfactant complexes (PG, black bars) or plasmid DNA-gemini surfactant-DOPE nanoparticles (PGL, white bars). Data is also presented for plasmid DNA with (white) and without (black) DOPE, and Lipofectamine.

FIG. 3 is a graph showing mean cell viability (n=4, error bars indicate standard deviation) of cells transfected with plasmid DNA-gemini surfactant-DOPE nanoparticles (white bars) n=4, bars=SD). Data is also presented for untransfected cells and cells transfected with plasmid DNA only (black bars), n=4, error bars indicate standard deviation, and cells transfected with Lipofectamine (gray bar). Columns connected by a solid line show no significant difference, those connected with a dashed line show a significant difference (p<0.01).

FIGS. 4A and 4B are graphs showing molar ellipticities for (A) plasmid DNA-gemini surfactant complexes and (B) plasmid DNA-gemini surfactant-DOPE complexes, respectively, for the 245 nm CD signal (hashed bar=plasmid DNA, gray bars=plasmid DNA-gemini surfactant complexes, white bars=plasmid DNA-gemini surfactant-DOPE complexes) compared to observed transfection efficiencies (black bars) expressed as ng luciferase per 20,000 cells.

FIG. 5A is a graph showing the pH dependence of the hydrodynamic radii for gemini surfactants having N-substituted spacers. The imino-substituted compound (⋄) shows a significant transition in size at pH 6.3, while the aza-substituted compounds show significantly less pH dependence. FIG. 5B is a graph showing the pH dependence of the hydrodynamic radius of the PGL complexes. FIG. 5C is a graph showing the pH dependence of zeta potential of the PGL nanoparticles.

FIG. 6A is a graph showing small angle X-ray scattering (SAXS) profiles for the plasmid DNA-gemini surfactant-DOPE complexes formed with surfactants 12-2-12, 12-5N-12, 12-8N-12, 12-7N-12, and 12-7NH-12.

FIG. 6B is a graph showing SAXS profiles for the plasmid DNA-gemini surfactant-DOPE complexes complexes formed with surfactants 12-3-12, 12-4(OH)₂-12, 12-E0₁-12, and 12-7NH-12.

FIG. 7 is a graph showing the surface tension (y) and observed enthalpies of dilution (ΔH_{obs}) for the 12-8N-12 surfactant.

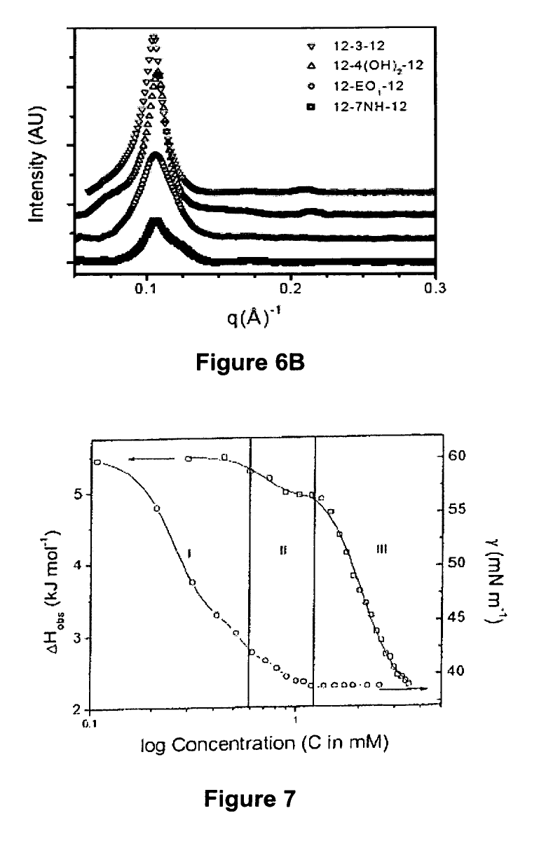

FIG. 8 shows the synthesis scheme for asymmetric tail, pyrene-substituted gemini surfactants.

FIG. 9A shows the ultraviolet absorbance spectra of an asymmetric tail, pyrene-substituted gemini surfactant. FIG. 9B shows the fluorescence emission spectra of an asymmetric tail, pyrene-substituted gemini surfactant.

### DETAILED DESCRIPTION

### I. Introduction

Gemini surfactants are a family of compounds generally characterized by having a long hydrocarbon chain (i.e., tail) connected to an ionic head group, which is connected via a spacer to another ionic head group connected to a long hydrocarbon chain (tail). The structures of gemini surfactants range from the simple m-s-m type (where m is the number of alkyl carbon atoms in the tail and s is the number of alkyl carbon atoms in the spacer [10-12], to more complex peptide-based [13] and carbohydrate-based [14-20] surfactants. Some gemini surfactants form a complex with biologically active agents (e.g., nucleic acids), which complex can be transfected into a cell.

In one aspect, the present compositions, delivery systems, and methods relate to spacer-substituted gemini surfactants, and methods of use, thereof. The spacer substitutions to be described enhance the efficacy of the substituted surfactants as nucleic acid transfection agents. In some embodiments, the substitutions are N-substitutions, such as azo or imide substitutions in the spacer. In other embodiments, the substitutions are O-substitutions, such as hydroxyl, ether, carboxyl, or ether substitutions.

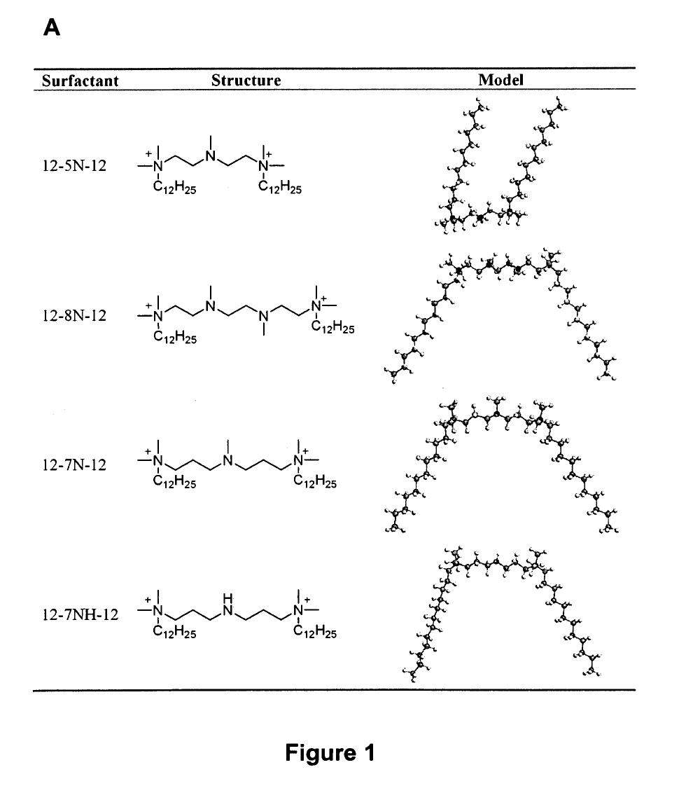

In some embodiments, the substituted-spacer gemini surfactants are N-substituted, having N—CH₃ (aza) or N—H (imino) substituents (groups, moieties) present in the spacer (FIG. 1A-C). The presence of an amine functional group in the spacer increases binding to nucleic acids, increases nucleic acid compaction, and increases nucleic acid transfection efficiency. Surfactants having N-substitutions in the spacer may be referred to, herein, as “N-substituted-spacer surfactants,” or “N-substituted surfactants,” for brevity. Particular substituted surfactants may be referred to as azo-substituted or imino-substituted, with the understanding that the substitutions are in the spacer.

In another embodiment, the spacer-substituted gemini surfactants are O-substituted, having —O— (ether) or —OH⁻ (hyrdroxyl) substituents/groups in the spacer (FIG. 1D-E). Surfactants having O-substitutions in the spacer may be referred to, herein, as “O-substituted-spacer surfactants,” or “O-substituted surfactants,” for brevity. Particular substituted surfactants may be referred to as hydroxyl-substituted or ether-substituted, with the understanding that the substitutions are in the spacer.

In experiments performed in support of the present compositions, systems, and methods, different N-substituted and O-substituted gemini surfactants were prepared and tested for their ability to enhance nucleic acid transfection efficiency (i.e., plasmid DNA encoding luciferase) into mammalian cells (Examples 1 and 2). Physical properties of transfection complexes were studied using, e.g., circular dichroism, small angle x-ray scattering (SAXS) measurements, and/or other measurements (Examples 3-6). Correlations between surfactant structure, complex morphology, and transfection efficiency are discussed.

In another embodiment, the modified gemini surfactants are asymmetric gemini surfactants in which one of the hydrocarbon tails comprises a pyrene moiety (FIG. 1F, Example 7). The asymmetric pyrene-substituted surfactants are particularly well-suited for use as a “tracer” for tracking the migration of transfection complexes in a subject, monitoring their entry into cells, and detecting the release of an attached biologically active agent. These surfactants may be referred to herein as “asymmetric surfactants,” or “pyrene-substituted surfactants,” for brevity, with the understanding that the substitutions are in the hydrocarbon tail. Experiments performed in support of the present compositions, systems, and methods are described, below.

### II. Transfection Efficiency and the Viability of Transfected Cells

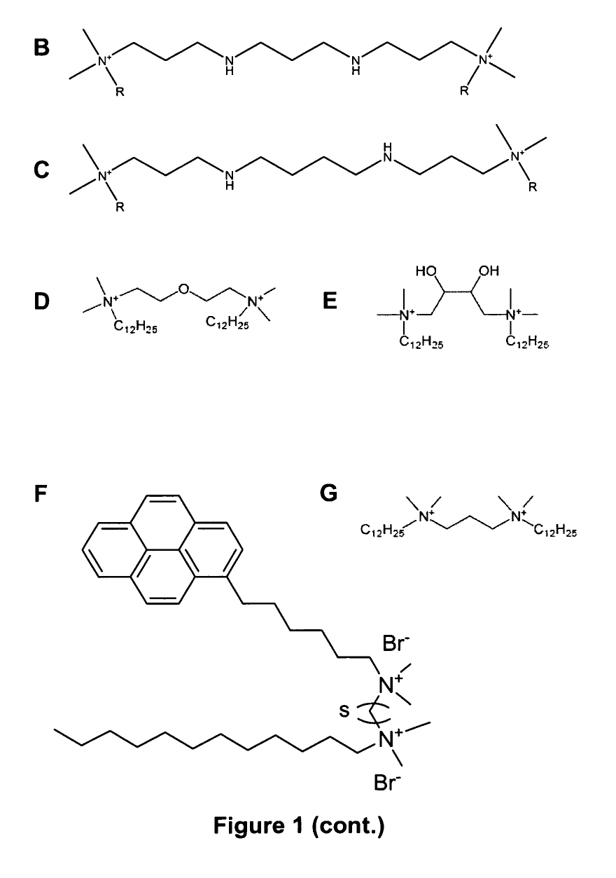

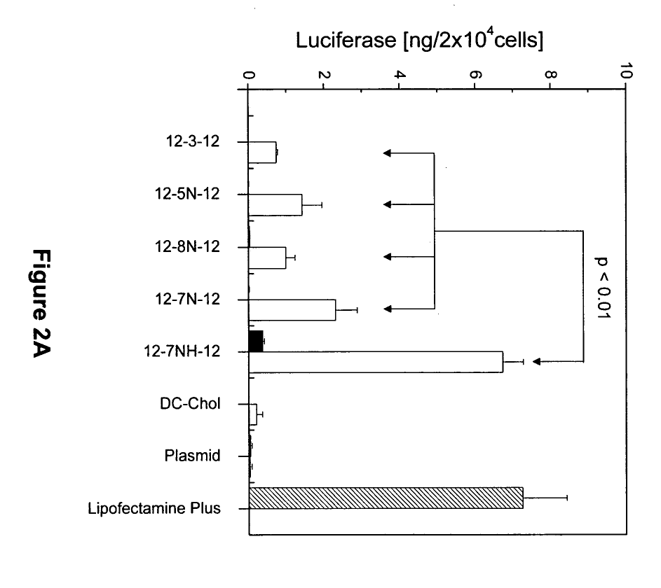

Transfections were performed as described in Example 2. The transfection efficiencies of different plasmid DNA-gemini (PG) and plasmid DNA-gemini-DOPE (PGL) complexes are shown in FIG. 2. The graph shows the relative amount of luciferase activity in cells following transfection with different plasmid DNA-gemini surfactant complexes (i.e., PG, black bars) or plasmid DNA-gemini surfactant-1,2 dioleyl-sn-glycero-phosphatidylethanolamine (DOPE) complexes (i.e., PGL, white bars). For comparison, results are shown for transfection with (i) the transfection reagent, 3-β[N—(N′,N′-dimethylaminoethane)-carbamoyl] cholesterol (i.e., DC-Chol) with plasmid DNA and with (white) or without (black) DOPE; (ii) naked plasmid DNA with (white) or without (black) DOPE; (iii) the commercially available transfection reagent, LIPOFECTAMINE with plasmid DNA (shaded, far right), and (iv) the aforementioned m-3-m gemini surfactant 12-3-12, described in [25]. 12-3-12, which contains a 3-atom, unsubstituted alky linker.

As previously observed, the 12-3-12 surfactant produced low transfection efficiency when complexed with DNA in the absence of helper lipid (i.e., DOPE). The azo-substituted surfactants 12-5N-12, 12-8N-12, and 12-7N-12 demonstrated similarly low transfection efficiency compared to 12-3-12. Unexpectedly, the imino-substituted surfactant 12-7NH-12 showed a significantly higher level of transfection efficiency in the absence of added helper lipid (FIGS. 2A and 2B).

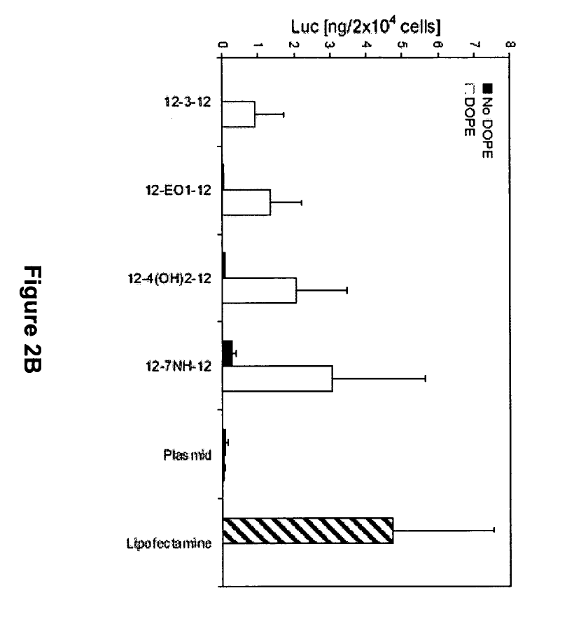

The addition of DOPE to the gemini surfactant-plasmid DNA complexes resulted in a significant increase in transfection efficiency with all tested gemini surfactants, consistent with results of previous studies [11, 27-30]. Helper lipids, such as DOPE, are believed to stabilize cationic liposomes to facilitate the release of DNA from complexes through membrane fusion and/or destabilization of the endosomal membrane [27, 31, reviewed in 32]. All three azo-substituted surfactants (12-5N-12, 12-8N-12, and 12-7N-12), as well as the imino-substituted surfactant (12-7NH-12), produced higher levels of transfection efficiency compared to the non-substituted gemini surfactant 12-3-12 (FIG. 3A). The hydroxyl-substituted surfactants (12-4(OH)₂-12) and, to lesser extent, the ether-containing surfactant (12-E0₁-12), also produced higher levels of transfection efficiency compared to the non-substituted gemini surfactant 12-3-12 (FIG. 3B).

Previous work relating to the 12-s-12 series of gemini surfactants, where s=3-8 atoms, showed that the 12-3-12 surfactant demonstrated the best transfection efficiency, while the 12-8-12 surfactant demonstrated the lowest [11]. Such results suggested that increasing spacer length decreased transfection efficiency. However, this relationship between spacer length and transfection efficiency did not appear to apply to the N-substituted surfactants. In particular, while the aza-substituted surfactants generally demonstrated increased transfection efficiency compared to 12-3-12, the most efficient aza-substituted surfactant was 12-7N-12. The aza-substituted surfactants with a shorter linker (i.e., 125N-12), and with a longer linker (i.e., 12-8N-12), were both less efficient in DNA transfection.

The highest transfection level of transfection efficiency were achieved with the PGL system containing the 12-7NH-12 surfactant (6.7±0.5 ng luciferase/2×10⁴ cells, p<0.01). This level of transfection efficiency was comparable to that of Lipofectamine Plus, the commercially available transfection agent.

Ideal transfection reagents are minimally toxic and/or disruptive to cells. Accordingly, the N-substituted surfactants were tested for toxicity in cultured cells. The viability of cells following transfection with either plasmid DNA-gemini surfactant-DOPE or Lipofectamine Plus are shown in FIG. 3. The aza-substituted surfactants 12-5N-12, 12-8N-12, and 12-7N-12 had similar toxicity compared to the Lipofectamine Plus reagent, with cell viability around 80% of the controls (i.e., no treatment or plasmid DNA alone; p>0.05). The imino-substituted surfactant 12-7NH-12 demonstrated slightly higher toxicity, with a cell viability of about 70% compared to Lipofectamine Plus (FIG. 3; p<0.01). The effect of the un-substituted surfactant 12-3-12 on cell viability was not statistically different from the control.

Overall, the cell viability observed with the 12-7NH-12 surfactant was not statistically significantly different from that of the 12-5N-12 and 12-8N-12 (p>0.05) surfactants. Therefore, within this series of compounds, cell toxicity does not appear to be a major concern and all the tested, substituted surfactants have sufficiently low toxicity to be useful as transfection reagents. The related surfactants, are likely to produce similar results in terms of toxicity. O-substituted surfactants were also shown to be useful as transfection agents.

### II. Physical Properties

Gemini surfactant-nucleic acid complexes were characterized using several methods (Examples 3 and 4). The relationship between the physical properties of a surfactant-nucleic acid complexes and transfection efficiency is discussed.

### A. SAMPLE HANDLING AND MEASUREMENTS

Surfactant solutions for physical analysis were generally prepared with water purified by using a Millipore Super-Q system. Critical micelle concentrations were determined at 25±0.1 C using both the electrical conductivity and surface tension methods as previously described [25]. The head group areas were determined from the surface excess concentration, Γ, in the usual manner according to equation:

a₀=(N_{A}Γ)⁻¹ (1)

where Γ is calculated from the Gibbs adsorption isotherm equation:

$$Γ = - \frac{1}{2.30 nRT} {( \frac{d γ}{d log C} )}_{T} \qquad ( 2 )$$

The parameters R and T have their usual meaning and n is a constant dependent upon the number of individual ions comprising the surfactant (n=3 for the m-s-m surfactants [51, 52].

Enthalpies were measured as previously described using a Calorimetry Sciences Corporation model 4200 isothermal titration calorimeter (ITC) with 1.3 mL cells [51]. Enthalpies of micellization were obtained from the difference in the observed enthalpies (of dilution) above and below the critical micelle concentration (cmc).

The density values required for the calculating the apparent molar volumes of the surfactants were obtained by using a Sodev model 02D vibrating tube flow densimeter following a technique previously described [53]. The apparent molar volumes (V_{φ}) of the aqueous surfactants were calculated using the equation:

$${V}_{φ} = \frac{M}{d} - \frac{1000 ( d - {d}_{0} )}{{mdd}_{0}} \qquad ( 3 )$$

where M is the molar mass of the solute, d and do are the densities of the solution and solvent in g·cm⁻³, respectively, and m is the molality of the solution in mol·kg-1.

The micelle hydrodynamic radii and micelle molecular weights were determined using a Malvern ZetaSizer NanoZS dynamic light scattering instrument. Aggregation numbers for the gemini surfactants were determined from these dynamic light scattering measurements based on a previously reported [51] molecular weight model relating micelle molecular weight to hydrodynamic radius, derived from the aggregation numbers of the CNTAB surfactant series obtained from fluorescence quenching methods.

### B. CIRCULAR DICHROISM

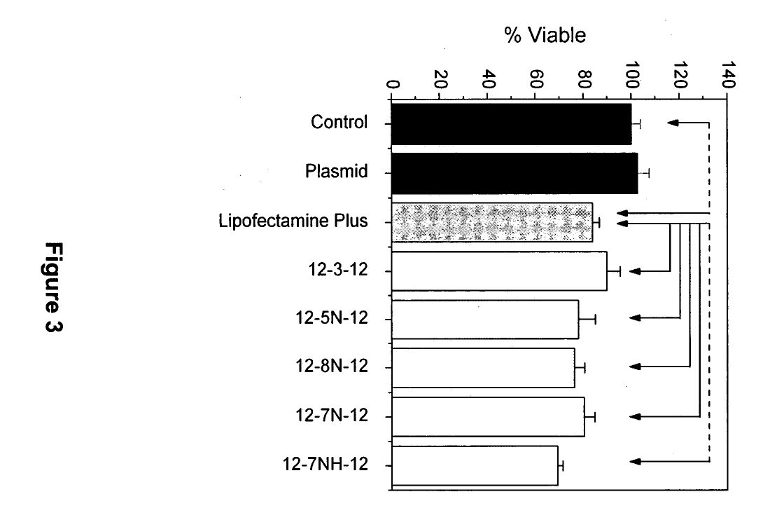

Circular dichroism (CD) measurements indicated that all gemini surfactants tested induced changes consistent with distortion of the DNA structure, similar to previous observations relating to cationic lipid-DNA complexes (FIG. 4). Both the positive signal at ˜280 nm and the negative signal at ˜245 nm undergo a red-shift upon complexation of DNA with the gemini surfactant in the presence or absence of DOPE. There was also a decrease in the positive ellipticity at 280 nm and an increase in the negative ellipticity at 245 nm; these effects being more pronounced for the plasmid DNA—gemini surfactant—DOPE complexes.

Similar changes in CD signal have been attributed to the complexed DNA adopting a more compact “ψ-form” [33, 34]. However, the changes shown in (FIG. 4) are more consistent with adoption of the B-DNA conformation, in which both the helicity and base stacking are perturbed by surfactant-mediated DNA compaction [35]. While no clear correlation was observed between transfection efficiency and changes in the CD spectrum, the plasmid DNA-12-7NH-12 surfactant-DOPE complex shows the greatest DNA distortion and the best transfection efficiency (FIG. 4B). This suggests that more efficient DNA compaction may explain, in part, the increase in transfection efficiency with the 12-7NH-12 surfactant.

### C. COMPLEX SIZE, pH DEPENDENCE, AND ZETA POTENTIAL

As noted above, it was unexpected that the 12-7N-12 and 12-8N-12 surfactants exhibited the highest and lowest transfection efficiencies, respectively, since the spacers differ in length by only a single methyl group. While the addition of a single aza substituent results in an increase in transfection efficiency compared to the unsubstituted surfactant, the addition of two aza substituents (as in the case of the 12-8N-12 surfactant; see, e.g., FIG. 1) decreased transfection efficiency. Moreover, previous results using non-substituted gemini surfactants demonstrated that shorter linkers were preferable, with the 12-3-12 surfactant being the most efficient transfection agent.

However, the present results using the N-substituted gemini surfactants suggest that transfection efficiency depends on (i) the impact of increased steric effects present in a di-substituted spacer group (i.e., with regard to the ability of the 12-8N-12 surfactant to bind DNA and form aggregates), and (ii) the equilibrium distance between nitrogen centers in the spacer compared to the distance between phosphate groups in a nucleic acid (which typically vary between 6.5 and 7.1 Å, depending upon the DNA sequence [36]).

Studies of polyamine-DNA complexes have determined that a spacing of 4.9 Å (equivalent to the trimethylene spacing in spermine) is suitable for interaction with adjacent phosphate groups in a nucleic acid molecule (i.e., DNA) [22, 37, 38]. The distance between nitrogen centers for the aza- and imino-substituted surfactants, obtained from the energy-minimized structures, are given in Table 1. The dimethylene spacing of ˜3.9 Å in the 12-5N-12 and 12-8N-12 surfactants is too short to readily interact with adjacent phosphate groups in DNA. The trimethylene spacing for the 12-7N-12 and 12-7NH-12 compounds is ˜5.1 Å, which is close to that of spermine, and allows better interaction with adjacent phosphate groups in. The interaction between the nitrogen centers in each tested N-substituted surfactant and DNA is illustrated by the energy minimized structures shown in Table 1. The nitrogen groups of the 12-7N-12 and 12-7NH-12 surfactants appear to have greater potential to interact with the phosphate groups of DNA.

TABLE 1
Distance between nitrogen centers for the aza- and imino-substituted
surfactants

| Surfactant | Atoms | d (Å) | Model |
|---|---|---|---|
| 12-5N-12 | N1-N2N1-N3N2-N3 | 3.90 7.52 3.88 | 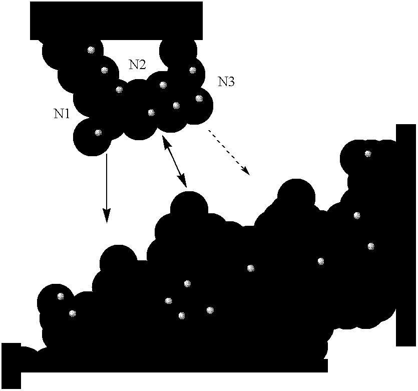 |
| 12-8N-12 | N1-N2N1-N3N1-N4N2-N3N2-N4N3-N4 | 3.86 7.4011.17 3.86 7.42 3.86 | 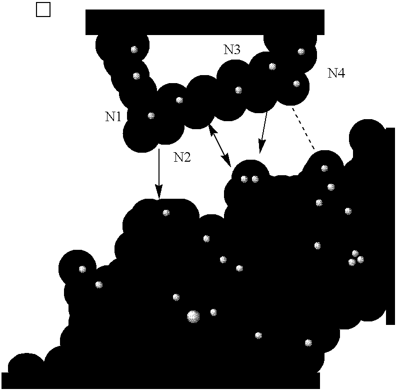 |
| 12-7NH-12 | N1-N2N1-N3N2-N3 | 5.0810.15 5.08 | 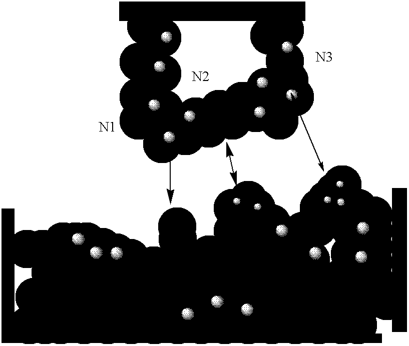 |
| 12-7N-12 | N1-N2N1-N3N2-N3 | 5.1410.18 5.14 | 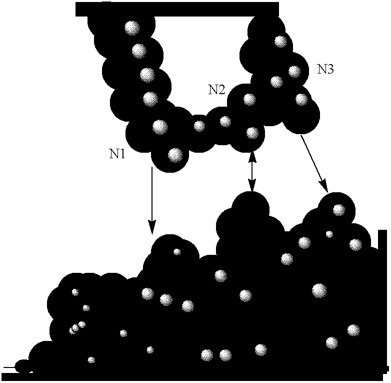 |

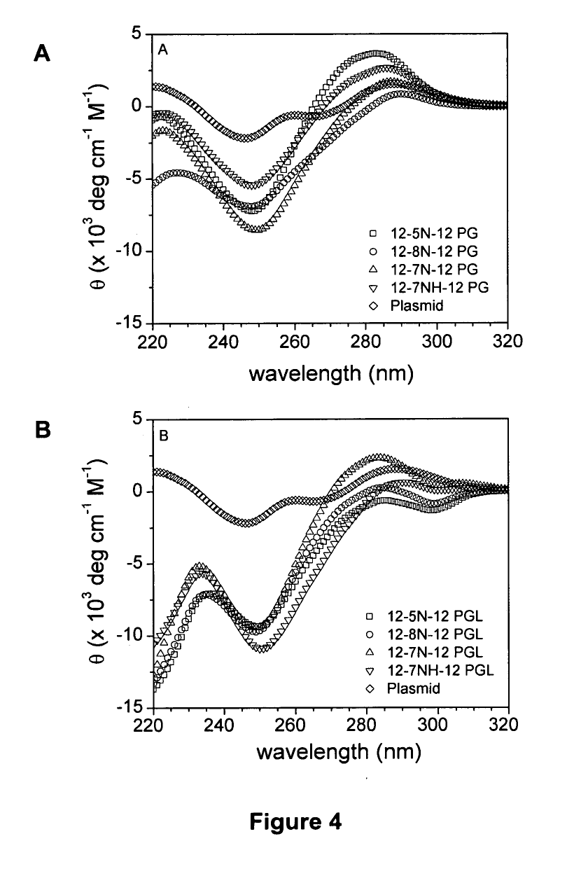

The increase in transfection efficiency observed for the 12-7NH-12 relative to the 12-7N-12 may, in part, relate to decreased steric hindrance. However, another explanation is the increased sensitivity to pH of the imino substituent compared to the aza substituent. FIG. 5A shows the pH dependencies for the hydrodynamic radii of the aza- and imino-substituted gemini surfactants. The 12-7NH-12 surfactant showed a significant change in aggregate size upon transition from basic to acidic pH, as a result of protonation of the imino group. While the observed change in aggregate size (from ˜2.5 to 3.7 nm radius) was not as significant as the vesicle to micelle transition observed in the sugar-based gemini surfactants [15-19], a clear structural change occurred on transition from neutral to acidic pH, which may aid in the release of DNA once the complex has been taken up by a cell.

Charged particles in solution or suspension attract a layer of counter-ions (ions of the opposite charge) which crowd the surface. Zeta potential is a measure (typically in mV) of the energy needed to shear a particle and its inner layer of counter-ions away from the bulk of the solution or suspension.

The pH dependence of complex size and zeta potential was determined for the PGL complexes containing either the 12-7N-12 or 12-7NH-12 surfactants (FIGS. 5B and 5C, respectively). The particle size remained essentially unchanged for complexes containing the 12-7N-12 surfactant, while the zeta potential increased as a function of decreasing pH which is consistent with the expected decrease in counterion (i.e. hydroxide) binding to the complex. Complexes containing the 12-7NH-12 surfactant also exhibit a pH-dependent transition in both particle size and zeta potential at ˜pH 5.5. Given the slightly acidic nature of the endosome [39], and that the transition of the present substituted surfactant-nucleic acid complexes occurs within this pH range, the incorporation of the pH sensitive imino-group in the spacer of surfactants may be particularly important for increasing transfection efficiency. Similar pH dependent transitions could be expected for Lipofectamine, based on the structure of the component DOSPA, and other polyamine-based lipids.

The majority of structures formed in cationic lipid-DNA systems are lamellar in nature (reviewed in [30, 31, 40]). However, other structures, including inverse hexagonal [28, 30] (linear and plasmid DNA [41, 42]) and cubic [43] (linear DNA) have been demonstrated. Increased transfection efficiency in cationic lipid-DNA complexes has been associated with the formation of inverse hexagonal bilayer structures [28]. More recently it has been observed that systems having lamellar structures exhibit reasonable transfection efficiencies provided the average membrane charge density is above a critical value [30, 44]. This is consistent with a recent study by Rosenzweig et al. [45], which demonstrated the formation of multilamellar structures from a series of O-alkyl dioleoylphosphatidylcholinium compounds, regardless of the structures formed by the hydrated lipids themselves.

Calculations of the average membrane charge density, σ_{M} for the present substituted surfactants show that, with the exception of the 12-5N-12 surfactant, σ_{M} is less than the critical value of 1.04×10⁻² e/Å² calculated by Safinya and co-workers (Table 1; [30, 44]). These data, in combination with the transfection efficiency data, suggest that the bilayer structures formed in these systems are not simply lamellar in nature, as further evidenced by the following small angle X-ray scattering measurements [30, 44].

### D. SMALL ANGLE X-RAY SCATTERING MEASUREMENTS

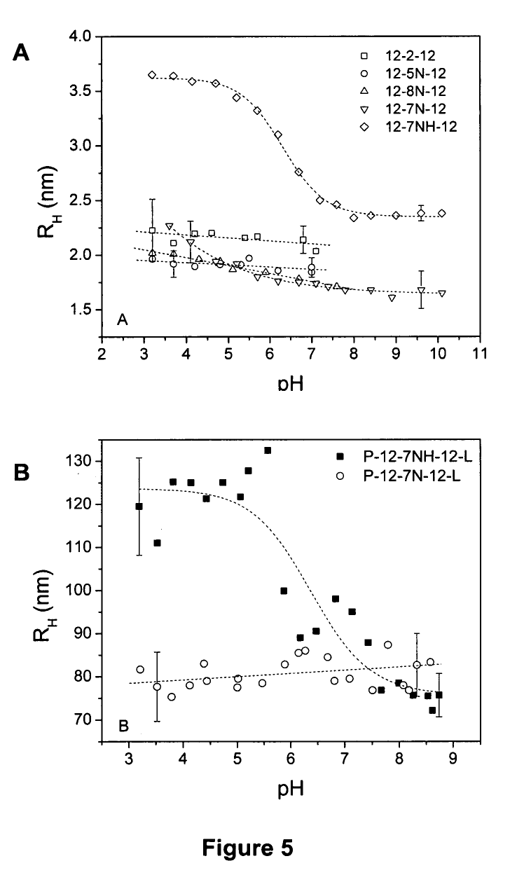

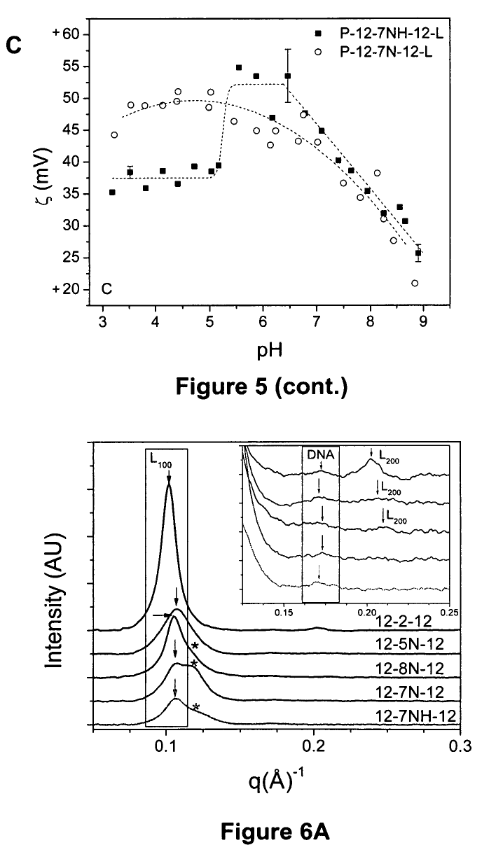

Small angle x-ray scattering (SAXS) measurements (i.e., SAXS profiles) obtained for complexes formed from plasmid DNA, the substituted gemini surfactants, and DOPE are shown in FIG. 6. The lipid bilayers in complexes formed with the 12-2-12 and 12-5N-12 surfactants exhibited scattering patterns (e.g., FIG. 6 and Table 2) consistent with a lamellar (L) phase (a d-spacing of 61.7 Å and 58.6 Å, respectively). Higher order reflections were nearly absent, which, along with the broad nature of the main scattering peaks, indicated a poorly ordered system. Weak peaks were also observed at q=0.172 Å⁻¹, which were attributed to scattering from adjacent DNA molecules with a d-spacing (d=27 dq) of 36.5 Å.

The 12-8N-12 surfactant showed a lamellar structure with d-spacing of 59.6 Å with weak scattering due to adjacent DNA molecules again being observed at q=0.170 Å⁻¹. A scattering peak, appearing as a broad shoulder on the main peak at 0.105 Å⁻¹ (indicated by “·” in FIG. 6) may indicate the presence of an additional phase.

The 12-7N-12 and 12-7NH-12 surfactants also exhibited a main scattering peak at q=0.105 and 0.106 Å⁻¹ (d=59.6 and 59.4 Å), respectively. However, second order peaks corresponding to a lamellar phase were not observed. Instead, additional scattering peaks were observed at q=0.117 and 0.116 Å⁻¹ for the 12-7N-12 and 12-7NH-12 surfactants, respectively. As before, a series of weaker peaks were detected, which may correspond to an (as yet) unidentified phase (see Table 2). It is evident from the relative magnitudes of the peaks at ˜0.116 Å⁻¹ that the second phase was present in a greater proportion than in the case of the 12-8N-12 surfactant.

TABLE 2
Head group areas (a₀) for a series of N-substituted surfactants, and
membrane charge densities (σ_{M}) and scattering peak positions
for the plasmid DNA-gemini surfactant-DOPE nanoparticles

| | | a₀^{a} | σ_{M}^{b} | q^{c} |
|---|---|---|---|---|
| Surfactant | n_{C,equiv} | (nm²/molecule) | (×10⁻² e/Å²) | (Å⁻¹) |
| 12-2-12 | 2 | 0.89 ± 0.05 | 1.43 | 0.102 (L), 0.172 |
| | | | | (DNA), |
| | | | | 0.203 (L) |
| 12-5N-12 | 5 | 1.30 ± 0.03 | 1.05 | 0.107 (L), 0.173 |
| | | | | (DNA), |
| | | | | 0.207 (L) |
| 12-8N-12 | 8 | 1.95 ± 0.13 | 0.780 | 0.105 (L), 0.119, |
| | | | | 0.169 (DNA), |
| | | | | 0.210 (L) |
| 12-7N-12 | 7 | 2.06 ± 0.20 | 0.748 | 0.083, 0.097, |
| | | | | 0.105, 0.117, |
| | | | | 0.134, 0.174 |
| 12-7NH-12 | 7 | 1.56 ± 0.04 | 0.920 | 0.083, 0.094, |
| | | | | 0.106, 0.116, |
| | | | | 0.171 |

^{a}From reference [24].
^{b}Calculated based on the equation σₘ = eZN_{cl}/(NₙₗAₙₗ + N_{cl}A_{cl}) [30, 44], where Z is the valence of the gemini surfactant (=2), Aₙₗ and A_{cl}, are the head group areas of DOPE and the gemini surfactant, respectively, and Nₙₗ and N_{cl} are the number of mols of DOPE and gemini surfactant. Aₙₗ = 46 Å², from reference [48].
^{c}For the plasmid DNA-gemini surfactant-DOPE systems. The letters in parenthesis indicate the phase associated with the observed scattering peak.

These results are consistent with recent SAXS results for the 12-3-12, 16-3-16, and 18:1-3-18:1 gemini surfactant-plasmid DNA-DOPE systems, which demonstrate the ability of these systems to adopt lamellar, inverse hexagonal, and cubic structures, depending on the relative ratios of surfactant, DNA, and DOPE [46, 47].

FIG. 6B is a graph showing SAXS profiles (Beamline X21A, NSLS, BNL) performed to determine the arrangement of lipid bilayers in the plasmid DNA-gemini surfactant-DOPE complexes formed with surfactants 12-3-12, 12-4(OH)₂-12, 12-EO₁-12, and 12-7NH-12. While complexes formed with surfactant 12-3-12 had a predominantly hexagonal morphology, surfactants 12-4(OH)₂-12 and 12-E0₁-12, with the hydrophilic substituents, exhibited scattering patterns consistent with mixed lamellar, hexagonal, and cubic phases which indexes to approximately Pn3m phase 3. Such observations suggested that certain gemini surfactants appear to form multiple phases, and have greater polymorphism, thereby making the resulting complexes more amenable to membrane fusion with endosomal lipids, enabling a nucleic acid to be released more readily.

### F. CONCLUSION

A series of aza- and imino-substituted gemini surfactants designed for use as nucleic acid transfection agents were synthesized and the resulting transfection complexes characterized. The gene transfer potential of these compounds was demonstrated by measuring transfection efficiencies in cultured cells.

The incorporation of aza- and imino-substituents within the spacer group enhanced the transfection efficiency of gemini surfactants, with the aza-substituted surfactants, 12-7N-12 and 12-5N-12, and the imino-substituted surfactant, 12-7NH-12, all resulting in enhanced transfection efficiency compared to the un-substituted surfactant, 12-3-12. In particular, incorporation of an imino group in the structure of the 1,9-bis(dodecyl)-1,1,9,9-tetramethyl-5-imino-1,9-nonanediammonium dibromide surfactant (12-7NH-12) resulted in a statistically significant (p<0.01), 9-fold increase in transfection compared to an un-substituted gemini surfactant and a 3-fold increase compared to the corresponding aza-substituted compound. A pH-dependent transition in size and zeta potential was observed to occur at pH 5.5 for complexes formed from the 12-7NH-12 compound. Small angle X-ray scattering (SAXS) results show weakly ordered structures and the presence of multiple phases.

The physical characterization of N-substituted surfactant-nucleic acid complexes indicated that the enhanced transfection efficiency may result from the formation of weakly ordered structures or the presence of multiple phases (other than hexagonal) that facilitate membrane fusion and the release of the complexed nucleic acid upon uptake by the cell. The distance between nitrogen centers within the spacer group may also impact the efficiency of transfection, pointing to the importance of structural features in both the surfactant and nucleic acid in the formation of an efficient transfection complex.

The structures of two related gemini surfactants are shown in FIGS. 1B and 1C. FIG. 1B shows the structure of 1,13-bis(dodecyl)-1,1,13,13-tetramethyl-5,9-diimino-1,13-tridecanediammonium dibromide (i.e., 12-13(NH)₂-12), which is related to that of 12-7NH-12. FIG. 1C shows the structure of 1,14-bis(dodecyl)-1,1,14,14-tetramethyl-5,10-diimino-1,14-tetradecanediammonium dibromide, which is related to 12-7NH-12 and to spermine. These substituted gemini surfactants are expected to have transfection efficiencies similar to 12-7NH-12.

The incorporation of ether and hydroxyl-substituents/groups within the spacer also enhanced the transfection efficiency, with the 12-4(OH)₂-12 and particularly 12-E0₁-12 surfactant resulting in enhanced transfection efficiency compared to 12-3-12.

The results further suggest that other charged groups may be substituted for azo, imino, hydroxyl, and ether groups, such as keto, ester, carboxyl, amino, guanidine, thiol, phosphodiester, sulfodiester, nitro, chloro, fluoro, and the like. Thus, gemini surfactants substituted with other pH-sensitive or polar functional groups are likely to produce acceptable results. Moreover, the methyl group of an aza spacer, or the hydrogen atom of an imino spacer, can be replaced by amino acids or short peptides.

The exemplified biologically active agent was DNA; however, the present compositions and methods are expected to work in combination with most any nucleic acid. Other biologically active agents for use according to the present methods include but are not limited to nucleic acid analogs and derivatives, such as synthetic nucleic acids containing thiodiester bonds, nucleoside analogs, 5′ and 3′ modifications, and modified nucleosides; and/or nucleic acid attached to proteins, carbohydrates, lipids, fluorescent and other detection groups, and the like.

### III. Asymmetric Gemini Surfactants

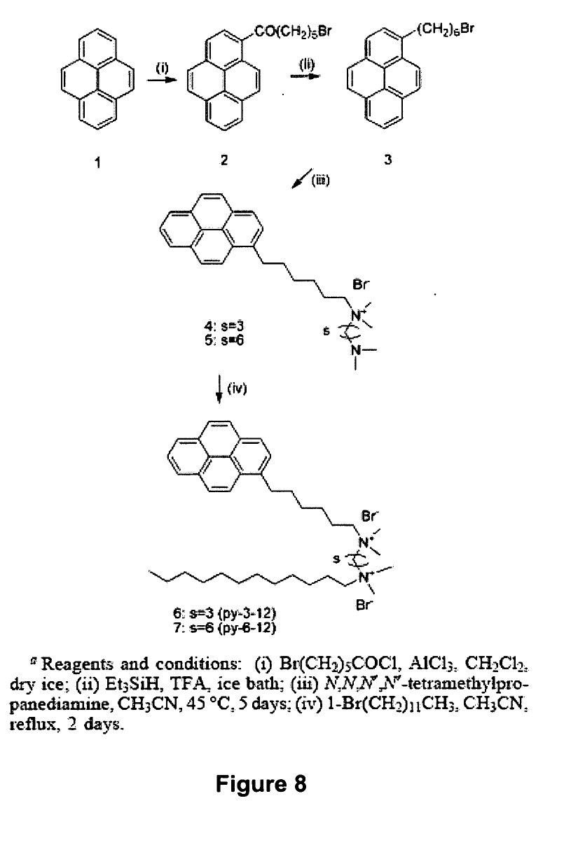

Two asymmetric gemini surfactants (py-3-12 and py-6-12) were synthesized in which a pyrene ring was substituted into one of the alkyl chains (i.e., tails; FIG. 1G; FIG. 8; Example 7). The pyrene moiety (group) has a characteristic absorption and emission spectra that allow it to be detected using e.g., real-time fluorescence microscopy. By substituting only one tail of the gemini molecule with a pyrene moiety, leaving the other tail undisturbed (i.e., as a comparatively simple hydrocarbon), the resulting asymmetric surfactant readily interacts with other gemini surfactants, including but not limited to the unsubstituted surfactant 12-3-12 and the N-substituted and O-substituted surfactants described above. In this manner, a gemini surfactant-DNA complex can be “tagged” or “labeled” with a suitable small amount of a pyrene-substituted gemini surfactant to allow the tracking of gemini surfactant-DNA transfections complexes into the body and into cells.

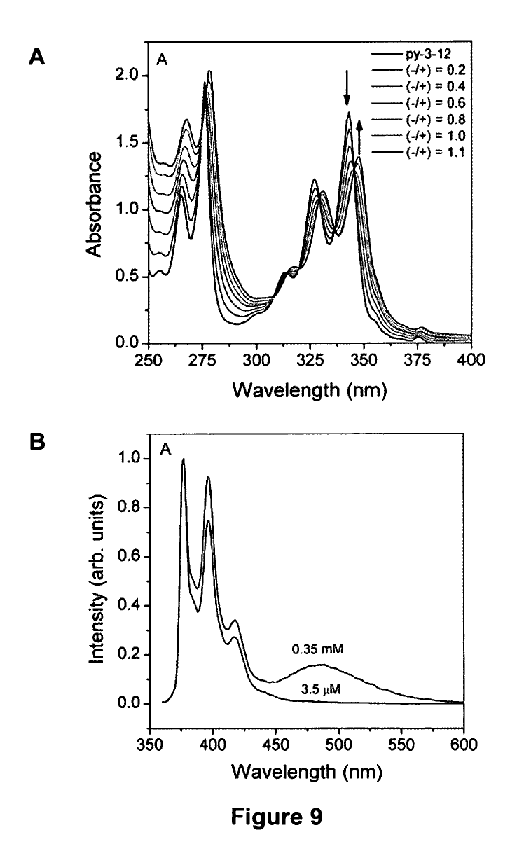

The absorbance spectrum for an exemplary py-3-12 is shown in FIG. 9A. Similar to a series of peptide substituted pyrene compounds described in [51], the S1-S0, S2-S0, and S3-S0 transitions have lambdaₘₐₓ values of 376, 343, and 276 nm, respectively. The broadening of the absorbance bands of pyrene, the red shift in the absorbance band, and the observed hyperchromism in the presence of DNA are evidence of complexed pyrene compounds Quantitative estimates of the binding constants between py-s-12 surfactants and DNA were obtained by monitoring changes in the UV spectrum of the pyrene moiety. The binding constant for py-3-12 and DNA was estimated to be 5.9×10³ L mol⁻¹ and that of py-6-12 was 1.9×10⁴ L mol⁻¹. The stronger binding of py-6-12 to DNA implied that the distance between the cationic head groups is more suitable to electrostatically bind to DNA.

Fluorescence emission spectra and fluorescence lifetimes were measured to confirm that the attachment of the pyrenyl moiety to the gemini surfactant did not result in any unusual photochemical effects. The fluorescence spectrum of py-3-12 at 3.5 μM (<cmc) is shown in FIG. 9B and is characteristic of pyrene in monomer form. There are three significant peaks at 377, 398, and 420 nm, which can be assigned to I₁, I₄, and I₅, respectively, in excellent agreement with results obtained for a series of peptide substituted pyrene derivatives described in [51]. Fluorescence spectra of py-s-12-DNA complexes were obtained by titrating 0.357 mM DNA into about 4.8 μM py-s-12.

There was a marked decrease in fluorescence emission of the monomer surfactant as DNA concentration is increased that may result from: (i) quenching of the pyrene fluorescence by DNA bases and/or (ii) quenching as a result of pyrene excimer formation. The concomitant emission peak at 480 nm, increases in intensity with increasing amounts of DNA until most of the surfactant was in complexed form. At a concentration of 0.35 mM (>cmc), a peak characteristic of pyrene excimers was also observed at 480 nm. The 480 nm emission peak that appears upon complexation of pyrene-substituted gemini surfactants with DNA can serve as a useful emissive probe for studying DNA trafficking in cellular transfection experiments, providing an opportunity to explore the mechanistic routes of transfection of DNA.

Accordingly, in some aspects, the present compositions and methods feature a delivery system for a biologically active agent comprising a gemini surfactant in admixture with a biologically active agent, such as a nucleic acid, and further comprising an amount of asymmetric, pyrene-substituted gemini surfactants (e.g., py-3-12 and py-6-12) to allow the monitoring or tracking of the admixture in a subject, or even into cells.

One skilled in the art will recognize that other pyrene-substituted gemini surfactants are likely to produce similar results. The amount of pyrene-substituted gemini surfactant present in the admixture should be sufficient to produce the 480 nm peak. In some embodiments, the amount is less than about 0.01, less than about 0.1, less than about 1, or even less than about 10% (wt/wt) of the total surfactant in the admixture.

### IV. Formulations, Dosages, and Treatment

The invention provides a method of delivering biologically active agents by preparing the delivery system (the gemini surfactant-biologically active agent complex as described above) and administering the delivery system topically to the skin or mucosal membrane.

Most preferably, the biologically active agents for use with the present invention are nucleic acids, plasmid DNA, DNA vaccines, and oligonucleotides. Further, the delivery system can be used for localized (intradermal and intramucosal), or systemic (transdermal or transmucosal) delivery, as well as for sustained release in or beneath the skin or mucosal membrane, namely, the epithelial membranes which line the oral cavity, the nasal, bronchial, pulmonary, trachea and pharynx airways; the otic and ophthalmic surfaces; the urogenital system, including the prostate, the reproductive system; the gastrointestinal tract including the colon and rectal surfaces; and the surface membranes or cell structures of the mucosal membrane at a subject's targeted site.

For this purpose, various formulations can be used for administration of the delivery system to the skin or mucosal membrane. Such formulations, whether pharmaceutically acceptable preparations or devices, preferably maintain contact with the skin or mucosal membrane. As formulations of the delivery system may lose some activity with aging, they can be either stabilized or generated fresh for administration.

Creams, Lotions, Pastes, Ointments, Foams—The delivery system may be incorporated into lipid formulations, emulsions, suspensions, creams, lotions, pastes, ointments or foams. Ointments or creams can be formulated with an aqueous or oily base with the addition of suitable thickening and/or gelling agents. Such bases may include water and/or an oil such as liquid paraffin or a vegetable oil such as peanut oil or castor oil. An exemplary base is water. Thickening agents which can be used according to the nature of the base include aluminum stearate, hydrogenated lanolin, and the like. Further, lotions can be formulated with an aqueous base and will, in general, include one or more of the following: stabilizing agents, emulsifying agents, dispersing agents, suspending agents, thickening agents, coloring agents, perfumes, and the like. Ointments and creams can also contain excipients, such as starch, tragacanth, cellulose derivative, polyethylene glycols, silicones, bentonites, silicic acid, and talc, or mixtures thereof. Lotions may be formulated with an aqueous or oily base and will, in general, also include one or more of the following: stabilizing agents, emulsifying agents, dispersing agents, suspending agents, thickening agents, coloring agents, perfumes, and the like. Foams may be formed with known foaming or surface active agents.

Gels and Liquids—The delivery system may be incorporated into gels, aqueous or non-aqueous solutions, sprays, mists or aerosols. Gels may be formed by mixing the delivery system with gelling agents such as collagen, pectin, gelatin, agarose, chitin, chitosan and alginate. The delivery system may be incorporated into liquids, formulated as topical solutions, aerosols, mists, sprays, drops and instillation solutions for body cavities. Administration of the delivery system to the mucosal membrane may be performed by aerosol, which can be generated by a nebulizer, or by instillation.

Coated Substrates—Substrates such as dressings, packings, films or meshes can be coated with the delivery system and used directly on the skin or mucosal membrane.

Transdermal Patch—Transdermal patches incorporating the delivery system can be attached to the skin or mucosal membrane to provide controlled, sustained release of the biologically active agent in or within the skin or mucosal membrane.

The delivery system may be administered alone, or with suitable non-toxic, pharmaceutically acceptable carriers, diluents and excipients suitable for topical application, as are well known in the art, see for example, Merck Index, Merck & Co., Rahway, N.J.; and Gilman et al., (eds) (1996) Goodman and Gilman's: The Pharmacological Bases of Therapeutics, 10^{th} Ed., McGraw-Hill. Carriers, diluents, excipients or supplements as used in the pharmaceutical compositions of the present invention are meant to refer to vehicles which are biocompatible, pharmaceutically acceptable, and suitable for administration to the skin or mucosal membrane. For standard dosages of conventional pharmacological agents, see for example, the U.S. Pharmacopeia National Formulary (2003), U.S. Pharmacopeial Convention, Inc., Rockville, Md. All agents must be non-toxic and physiologically acceptable for the intended purpose, and must not substantially interfere with the activity of the biologically active agent.

The dosage of the delivery system depends upon many factors that are well known to those skilled in the art, for example, the particular form of the biologically active agent within the delivery system, the condition being treated, the age, weight, and clinical condition of the recipient patient, and the experience and judgement of the clinician or practitioner administering the therapy. A therapeutically effective amount provides either subjective relief of symptoms or an objectively identifiable improvement as noted by the clinician or other qualified observer. The dosing range varies with the biologically active agent within the delivery system used, its form, and the potency of the particular agent.

It will be appreciated that the delivery system can be used with any gene having therapeutic effects for the above skin disorders and metabolic diseases.

Further embodiments of the compositions, systems, and methods will be apparent to the skilled artisan upon reading the disclosure. The following examples illustrate the compositions, systems, and methods but are in no way intended to be limiting.

### EXAMPLES

The following examples are provided to further illustrate the compositions, systems, and methods.

### Example 1

### Synthesis of N-Substituted and O-Substituted Gemini Surfactants

The synthesis of the surfactants used in this study has been previously described [24, 25]. Aqueous solutions (1.5 mM) of the gemini surfactants were prepared and filtered through 0.2 μm Acrodisc® filters (Pall Gelman, Ann Arbor, Mich.). Lipid vesicles were prepared by using sonication techniques. 1,2 dioleyl-sn-glycero-phosphatidylethanolamine (DOPE; Avanti Polar Lipids, Alabaster, Ala.) and α-tocopherol (Spectrum, Gardena Calif.), in 1:0.2 weight ratios, were dissolved in 100% ethanol (Commercial Alcohols Inc., Brampton, ON), and deposited as a thin film in a round bottom flask. The lipid was vacuum-dried overnight, to remove traces of solvent, and resuspended at 2 mM concentration in 9.25% w/v isotonic sucrose (Spectrum, Gardena, Calif.) solution (pH 9) by sonication, and then filtered through 0.45 μm Acrodisc® filters giving DOPE vesicles with an average size of 124±3 nm (n=3).

### Example 2

### Transfection Assay

COS-7 African Green monkey kidney fibroblast cells (ATCC CRL1651) were prepared for transfection in antibiotic and 10% fetal bovine serum supplemented minimal Eagle's medium (MEM). The day before transfection, 5×10⁴ cells/well were seeded in 24-well plates (Greiner Labortechnik GmbH, Germany). The supplemented MEM was changed to MEM one hour prior to transfection.

The pMASIA.Luc plasmid DNA was used at a concentration of 0.2 μg/well for transfection. The transfection mixtures were prepared as follows: 0.2 μg of plasmid DNA was mixed with aliquots of gemini surfactant solution to obtain a plasmid DNA:gemini surfactant charge ratio of 1:10 and incubated at room temperature for 15 minutes. To this mixture, 25 μL of DOPE liposomes were added. The transfection mixtures were incubated for 30 minutes at room temperature prior to transfection and added to the cells, dropwise. The plates were incubated for 5 hours at 37° C. in a CO₂ incubator. Dc-Chol (30-[N—(N′,N′-Dimethylaminoethane)-carbamoyl]cholesterol) was used for comparison and, as a positive control, cells were also transfected with Lipofectamine Plus™ Reagent (Invitrogen Life Technologies, Carlsbad, Calif.), according to the manufacturer's protocol, with 0.2 μg of plasmid DNA per well.

Luciferase production was quantified using the Promega Steady-Glo™ luciferase assay system. Luminescence was monitored using a Turner Biosystems (Sunnyvale, Calif., USA) Reporter microplate luminometer. Statistical analysis was performed using ANOVA and Tukey's multiple comparison test. Signifcant differences were observed at alpha level of 0.05. Equal variances were tested using Bartlett's test, and normality by the D'Agostino & Pearson omnibus normality test.

### Example 3

### Physical Characterization

Determinations of size (hydrodynamic radius, R_{H}) and zeta potential (ζ) as a function of pH were carried out using a Malvern Instruments (Worcestershire, UK) ZetaSizer NanoZs with MPT-2 auto-titrator attachment. 10 mL of the desired sample was placed in the titration cell of the auto-titrator and samples were titrated (in 0.2-0.5 pH unit increments) with 0.1 M NaOH and 0.1 M HCl over the desired pH range. Size and zeta potential measurement were made in triplicate at each pH point, and the reported values represent the average±standard error. pH titrations of aqueous gemini surfactant solutions were made at a surfactant concentration of 2.00 mM, above the critical micelle concentration in all cases.

CD spectra were obtained by using an Applied Photo Physics π*180 instrument (Leatherhead, UK) with a 4 nm slit, at room temperature.

Solutions for size, zeta potential, and circular dichrosicm (CD) measurements were prepared, in larger volumes, as described above for the transfection experiments using a pGTmCMV.IFN-GFP plasmid DNA, previously constructed in our lab [1,1]. Solutions containing only surfactant and plasmid DNA were prepared by addition of an aliquot of stock plasmid DNA to the gemini surfactant solution to a charge ratio of 1:10 (DNA:surfactant).

### Example 4

### Small Angle X-Ray Scattering (SAXS)

SAXS measurements were carried out at beamline X21 at the National Synchrotron Light Source at Brookhaven National Laboratory. Beamline X21 is based on a wiggler source, with a helium gas-cooled Si(111) monochromator selecting the photon energy. The X-ray beam is focused by a toroidal mirror into the experimental station. Three pairs of precision slits, positioned at 4 m, 1 m, and 5 cm upstream of the sample, define the beam. The scattering pattern is recorded using a 13 cm Mar CCD detector (Mar USA, Evanston, Ill.) located 1.26 m (calibrated with the scattering pattern of silver behenate) downstream of the sample. The measurements were performed with 12 KeV x-rays and the data covered a q-range from 0.008 Å⁻¹ to 0.375 Å⁻¹. All beam paths are under vacuum, except at the sample position, where the sample is in air, and kapton windows terminate the vacuum beam paths. Samples were loaded into 1.5 mm capillaries (Charles Supper #15-BG, Natick Mass.) and data recorded at room temperature. All spectra were processed to remove background contributions by subtracting the scattering profile obtained for a water-filled capillary. Peak fitting was carried out using the open-sources software package “fityk” (available at http://www.unipress.waw.pl/fityk/; last accessed on Jul. 5, 2007) using multiple Gaussian functions.

Solutions for SAXS analysis were prepared by addition of appropriate amounts of stock surfactant, DNA, and DOPE solutions, followed by sonication (to ensure complete mixing) where appropriate. The pGTmCMV.IFN-GFP plasmid DNA was used in the SAXS studies. The final concentrations of DNA and DOPE were 0.075 mM (50 μg/mL) and 1.00 mM (0.75 mg/mL), respectively. The concentration of gemini surfactant was adjusted as needed to obtain the desired 1:10 DNA:surfactant charge ratio. Preliminary x-ray scattering studies indicated these concentrations to be too dilute to give good scattering signals; as a result, the samples were concentrated 5× by evaporation.

### Example 5

### Molecular Modeling

The energy minimized gas-phase structures (shown in FIG. 1) of the gemini surfactants were calculated using the MM2 force field in Chem 3D Pro v. 8.0 to a RMS gradient of 0.05. A model DNA fragment was constructed from crystallographic data (RCSB PDB file 194D [26]).

### Example 6

### Statistical Analysis

Statistical analysis was performed by one-way ANOVA and the groups were compared using Tukey's multiple comparison test using GraphPad Prism 4.01 software.

### Example 7

### Synthesis of Pyrene-Substituted Gemini Surfactants

Pyrene, 6-bromohexanoyl chloride, anhydrous aluminum chloride, triethylsilane, trifluoroacetatic acid, N,N,N′,N′-tetramethylpropanediamine, N,N,N′,N′-tetramethylhexanediamine, and 1-bromododecane were purchased from Aldrich Inc. All solutions were prepared using analytical-grade water obtained from a Millipore Milli-Q filtration system. The sodium salt of double-stranded (ds) salmon sperm DNA (about 2 kbp, as stated by the manufacturer, Sigma) was used as received. DNA stock solutions (usually 7.5 mM, i.e., 5.0 mg/mL) were prepared in distilled water (DNAse, RNAse free, Life Technologies) and homogenized by vortexing. The DNA concentration was obtained spectrophotometrically by measuring the absorption at 260 nm using the extinction coefficient (ε) 1.31×10⁴ M⁻¹ cm⁻¹. The synthesis is shown in FIG. 8 and detailed below. The italicized numbers in parenthesis refer to the compound numbers in the Figure.

### 5-Bromohexane-1-pyrene Ketone (2)

The synthesis was carried out as previously reported (49).

### 6-(1-Pyrenebromohexane) (3)

The synthesis was carried out as previously reported (50).

### Py-3(N-(3-dimethylaminopropyl)-N,N-dimethyl-6-(pyren-6-yl)-hexan-1-ammonium bromide) (4)

6-(1-Pyrene)bromohexane (3.8 g, 10.4 mmol) was added to N,N,N′,N′-tetramethylpropanediamine (2.2 mL, 13.2 mmol) in 80 mL of anhydrous acetonitrile. The mixture was stirred and maintained at 45° C. for 3 days. The solvent was then reduced under vacuum to ˜10 mL, and a white precipitate formed upon the addition of diethyl ether. The crude product was recrystallized from acetonitrile and diethyl ether and yielded a white solid (2.0 g, 39%). ¹H NMR (CDCl₃, 500 MHz) δ (ppm): 8.194-7.864 (m, 9H, py-H), 3.598 (broad, 2H, ⁺N—CH₂), 3.399-3.356 (m, 4H, py-CH₂ and CH₂N⁺), 3.231 (s, 6H, +N(CH3)2), 2.650 (broad, 2H, CH₂—N), 2.401 (s, 6H, N(CH₃)₂), 2.011-1.420 (m, 10H, (CH₂)₄ and β-CH₂).

### Py-6(N-(6-dimethylaminohexyl)-N,N-dimethyl-6-(pyren-6-yl)-hexan-1-ammonium bromide) (5)

6-(1-Pyrene)bromohexane (1.2 g, 3.3 mmol) was added to N,N,N′,N′-tetramethylhexanediamine (0.8 mL, 4.1 mmol) in 50 mL of anhydrous acetonitrile. The mixture was stirred at 50° C. for 3 days, and a white solid formed on the flask wall. The solvent was then reduced under vacuum to ˜10 mL, and diethyl ether was added. The crude product was recrystallized from acetonitrile and diethyl ether and produced a light-yellow solid (0.56 g, 32%). ¹H NMR (CDCl₃, 500 MHz) δ (ppm): 8.192-7.867 (m, 9H, py-H), 3.502 (t, 2H, ⁺N—CH₂), 3.474-3.312 (4H, py-CH₂ and ⁺NCH2), 3.211 (s, 6H, ⁺N(CH₃)₂), 2.850 (broad, 2H, CH₂N), 2.640 (s, 6H, N(CH₃)₂), 1.897-1.458 (m, 16H, (CH₂)₄ on spacer and C6 chain).

### Py-3-12(N1-dodecyl,N1,N1,N3,N3-tetramethyl-N3-(6-(pyren-6-yl)-hexyl)propane-1,3-diammonium dibromide) (6)

Py-3 (2.0 g, mmol) and 1-bromododecane (2.2 mL, 9.0 mmol) were added to 80 mL of anhydrous acetonitrile. The mixture was heated under reflux for 2 days. The solvent was removed under vacuum, and the crude product was recrystallized from acetone and chloroform and dried overnight under vacuum and produced a final product of 2.9 g (yield 96%). ¹H NMR (CDCl₃, 500 MHz) δ (ppm): 8.183-7.874 (9H, m, py-H), 3.858 (4H, t, N—CH₂ (spacer)), 3.419 (6H, m, py-CH₂ and N—CH₂ (chain)), 3.278 (6H, s, N—CH₃ (py side)), 3.200 (6H, N—CH₃ (C12 side), 2.722 (2H, m, CH₂ (spacer)), 1.906-1.269 (28H, m, —CH₂—), 0.911 (3H, t, C—CH₃). Analysis: calculated for py-3-12.2H₂O (C₄₁H₆₄N₂Br₂.2H₂O): C, 63.07%; H, 8.78%; N, 3.59%. Found: C, 62.97%; H, 8.85%; N, 3.52%.

### Py-6-12(N1-dodecyl,N1,N1,N6,N6-tetramethyl-N6-(6-(pyren-6-yl)-hexyl)hexane-1,6-diammonium dibromide) (7)

Py-6 (0.55 g, 1.0 mmol) was added to 1-bromododecane (1.23 mL, 5.0 mmol) in 110 mL of acetonitrile. The mixture was stirred at 50° C. for 4 days. The solvent was then removed under vacuum. The product was recrystallized in diethyl ether and yielded an off-white solid (0.78 g, 97%). ¹H NMR (CDCl₃, 500 MHz) δ (ppm): 8.181-7.278 (9H, m, py-H), 3.732-3.675 (4H, m, N—CH₂ (spacer)), 3.675-3.370 (6H, m, py-CH₂ and N—CH₂ (chain)). 3.256 (6H, s, N—CH₃ (spacer), 3.177 (6H, s, N—CH₃ (C12 side)), 2.053-1.276 (36H, m, —CH₂—), 0.909 (3H, t, C—CH₃). Analysis: Calculated for (C₄₄H₇₀N₂Br₂): C, 67.16%; H, 8.97%; N, 3.56%. Found: C, 66.05%; H, 8.98%; N, 3.49%.

What is claimed is:

1. A delivery system for a biologically active agent comprising:
a gemini surfactant in admixture with a biologically active agent,
said gemini surfactant having head groups and a spacer linking said head groups, said spacer comprising a hydrophilic substituent in the spacer, wherein the delivery system, when in contact with skin or a mucosal membrane, provides a therapeutic effect.

2. The delivery system of claim 1, wherein the hydrophilic substituent is selected from the group consisting of aza, imino, hydroxyl, and ether.

3. The delivery system of claim 1, wherein the hydrophilic substituent is the result of an N-substitution or an O-substitution.

4. The delivery system of claim 1, wherein said spacer is N-substituted and the substituent is selected from aza and imino.

5. The delivery system according to claim 4, wherein said spacer is from 3-8 atoms in length and comprises an imino substituent.

6. The delivery system according to claim 5, wherein said spacer is 7 atoms in length.

7. The delivery system according to claim 6, wherein said gemini surfactant is 12-7NH-12.

8. The delivery system according to claim 6, wherein said gemini surfactant is 1,9-bis(dodecyl)-1,1,9,9-tetramethyl-5-imino-1,9,-nonanediammonium dibromide.

9. The delivery system according to claim 4, wherein said spacer is from 3-8 atoms in length and comprises an aza substituent.

10. The delivery system according to claim 9, wherein said spacer is 7 atoms in length.

11. The delivery system according to claim 10, wherein said gemini surfactant is 12-7N-12.

12. The delivery system according to claim 10, wherein said gemini surfactant is 1,9-bis(dodecyl)-1,1,5,9,9-pentamethyl-5-aza-1,9,-nonanediammonium dibromide.

13. The delivery system of claim 1, wherein said spacer is O-substituted and the substituent is selected from hydroxyl and ether.

14. The delivery system according to claim 13, wherein the substituent is hydroxyl.

15. The delivery system according to claim 14, wherein said gemini surfactant is 12-4(OH)₂-12.

16. The delivery system according to claim 13, wherein the substituent is ether.

17. The delivery system according to claim 16, wherein said gemini surfactant is 12-EO₁-12.

18. The delivery system according to claim 5, for use in the absence of added helper lipid.

19. The delivery system according to claims 1, further comprising DOPE.

20. The delivery system according to claim 1, wherein the gemini surfactant is a cationic surfactant.

21. The delivery system according to claim 20, wherein the gemini cationic surfactant is of a quaternary ammonium type.

22. The delivery system according to claim 1, wherein the gemini cationic surfactant has a hydrophobic tail comprising a C3-C30 alkyl group, linear or branched, saturated or unsaturated.

23. The delivery system according to claim 1, wherein the biologically active agent is a nucleic acid.

24. The delivery system according to claim 23, wherein the biologically active agent is DNA.

25. The delivery system according to claim 1, provided as a component of a pharmaceutical product.

26. The delivery system according to claim 1, wherein the delivery system is formulated with a pharmaceutically acceptable component to form a pharmaceutical composition.

27. The delivery system according to claim 1, further comprising an asymmetric gemini surfactant having a first head group and first tail, a second head group and second tail, and a spacer linking said first and second head groups, wherein the first tail comprises pyrene.

28. The delivery system according to claim 27, wherein the asymmetric gemini surfactant is py-3-12.

29. The delivery system according to claim 27, wherein the asymmetric gemini surfactant is py-6-12.

30. The delivery system according to claim 27, wherein the asymmetric gemini surfactant is present in a trace amount to allow the detection of the gemini surfactant in admixture with the biologically active agent.

31. The delivery system according to claim 30, wherein said detection is performed by measuring fluorescence at 480 nm.

32. A delivery system for a biologically active agent comprising:
an asymmetric gemini surfactant in admixture with a biologically active agent,
said asymmetric gemini surfactant having a first head group and first tail, a second head group and second tail, and a spacer linking said first and second head groups, wherein said first tail comprises pyrene,
wherein the delivery system, when in contact with skin or a mucosal membrane, provides a therapeutic effect.

33. The delivery system according to claim 32, wherein the asymmetric gemini surfactant is py-3-12.

34. The delivery system according to claim 32, wherein the asymmetric gemini surfactant is py-6-12.

35. The delivery system according to claim 32, wherein the biologically active agent is a nucleic acid.

36. The delivery system according to claim 35, wherein the biologically active agent is DNA.

37. The delivery system according to any one of claim 32, provided as a component of a pharmaceutical product.

38. The delivery system according of claim 32, wherein the delivery system is formulated with a pharmaceutically acceptable component to form a pharmaceutical composition.

39. A gemini surfactant having head groups and a spacer linking said head groups, said spacer comprising an N-substituted substituent selected from aza and imino.

40. The gemini surfactant of claim 39, wherein said spacer is from 3-8 atoms in length and comprises an imino substituent.

41. The gemini surfactant of claim 40, wherein said spacer is 7 atoms in length.

42. The gemini surfactant of claim 41, wherein the gemini surfactant is 12-7NH-12.

43. The gemini surfactant of claim 40, wherein said gemini surfactant is 1,9-bis(dodecyl)-1,1,9,9-tetramethyl-5-imino-1,9,-nonanediammonium dibromide.

44. The gemini surfactant of claim 39, wherein, said spacer is from 3-8 atoms in length and comprises an aza substituent.

45. The gemini surfactant of claim 44, wherein said spacer is 7 atoms in length.

46. The gemini surfactant of claim 45, wherein said gemini surfactant is 12-7N-12.

47. The gemini surfactant of claim 44, wherein said gemini surfactant is 1,9-bis(dodecyl)-1,1,5,9,9-pentamethyl-5-aza-1,9,-nonanediammonium dibromide.

48. A gemini surfactant having head groups and a spacer linking said head groups, said spacer comprising an O-substituted substituent selected from hydroxyl and ether.

49. The gemini surfactant of claim 48, wherein the gemini surfactant is 12-4(OH)₂-12.

50. The gemini surfactant of claim 48, wherein the gemini surfactant is 12-EO₁-12.

51. An asymmetric gemini surfactant having a first head group and first tail, a second head group and second tail, and a spacer linking said first and second head groups, wherein the first tail comprises pyrene.

52. The gemini surfactant of claim 51, wherein said spacer is from 3-6 atoms in length.

53. The gemini surfactant of claim 52, wherein said spacer is 3 atoms in length.

54. The gemini surfactant of claim 52, wherein the gemini surfactant is py-3-12.

55. The gemini surfactant of claim 51, wherein said spacer is 6 atoms in length.

56. The gemini surfactant of claim 55, wherein the gemini surfactant is py-6-12.

57. A method of treating skin disorders and metabolic diseases comprising:
contacting the skin or mucosal membrane of a subject with a delivery system comprising a gemini surfactant having head groups and a spacer linking said head groups, said spacer being N-substituted or O-substituted, in admixture with a biologically active agent in a topical formulation, wherein the delivery system, when in contact with skin or a mucosal membrane, provides a therapeutic effect.

58. The method according to claim 57, wherein the gemini surfactant is a gemini cationic surfactant.

59. The method according to claim 57, wherein the gemini cationic surfactant is of a quaternary ammonium type.

60. The method according to claim 57, wherein the gemini surfactant is selected from the croup consisting of 12-7NH-12, 12-7N-12, 12-4(OH)₂-12, and 12-EO₁-12.

61. A method for detecting a gemini surfactant-DNA complex in a subject, comprising:
adding to an admixture of a gemini surfactant and DNA an amount of an asymmetric, pyrene-substituted gemini surfactant,
exciting the pyrene-substituted gemini surfactant present in the admixture at a wavelength suitable for exiting pyrene, and
measuring the fluoresce of pyrene,
whereby the presence of the asymmetric, pyrene-substituted gemini surfactant in the admixture allows the detection of the admixture in the subject.

62. The method of claim 61, wherein the fluoresce of pyrene is measured at a wavelength of about 480 nm.

63. The method of claim 61, wherein the asymmetric, pyrene-substituted gemini surfactant is py-3-12.

64. The method of claim 61, wherein the asymmetric, pyrene-substituted gemini surfactant is py-6-12.
